(19) United States
(12) Patent Application Publication
(10) Pub. No.: US 2022/0249085 A1
(43) Pub. Date: Aug. 11, 2022

(54) TISSUE REPAIR DEVICE

(71) Applicant: Arthrex, Inc., Naples, FL (US)

(72) Inventors: Joshua J. Best, Naples, FL (US); Anthony A. Laviano, Fort Myers, FL (US); Matthew R. Johnson, Bonita Springs, FL (US); Zachary A. Ingwer, Naples, FL (US); Samuel B. Roque, Naples, FL (US); Kenneth T. Helenbolt, Naples, FL (US); Silas Wendell Holmes, JR., Elgin, SC (US); Andrew K. Osika, Naples, FL (US)

(73) Assignee: Arthrex, Inc., Naples, FL (US)

(21) Appl. No.: 17/623,733

(22) PCT Filed: Jul. 1, 2020
(86) PCT No.: PCT/US2020/040517

§ 371 Date: Dec. 29, 2021

Related U.S. Application Data
(60) Provisional application No. 62/869,806, filed on Jul. 2, 2019.

(51) Int. Cl. A61B 17/04

(52) U.S. Cl. CPC A61B 17/0401; A61F 2/0811; A61B 2017/0464; A61B 2017/0409

(57) ABSTRACT

Tissue repair devices that has a movable inserter configured to support a suture implant construct that is positioned for deployment and methods for using the devices.

### RELATED APPLICATION

This application claims priority to U.S. Provisional Application Ser. No. 62/869,806, filed on Jul. 2, 2019, and entitles Tissue Repair Device, the subject matter of which is herein incorporated by reference.

### BACKGROUND

This disclosure relates to a surgical device for repairing damaged tissue and a method for repairing the damaged tissue. Orthopedic procedures are often performed to repair musculoskeletal injuries, such as those sustained during sporting activities. Tears in the meniscus are known to be repaired by deploying implants on either side of the tear, tensioning suture between the implants to close the tear, and allowing it to heal.

### SUMMARY

This disclosure relates to a tissue repair device that comprises, inter alia, a fixed outer cannula that has a distal end and a proximal end opposite the distal end and a fixed length, the fixed length defining a depth stop; and a movable inserter received inside of the fixed outer cannula, the movable inserter may be slidable with respect to the fixed outer cannula, and the movable inserter may be configured to support a suture implant construct that is positioned for deployment.

In some embodiments, an actuator is received in the fixed outer cannula for sliding the movable inserter with respect to the fixed cannula; a depth limit selection feature is provided that corresponds to the fixed length of the fixed outer cannula; the depth limit selection feature is associated with a handle at the proximal end of the fixed outer cannula; and/or the depth limit selection feature is a rotating depth selector coupled to the proximal end of the fixed outer cannula.

In certain embodiments, the movable inserter includes a longitudinal slot providing access to an inside of the movable inserter; the width of the longitudinal slot is sized to accommodate a suture or sutures of the suture implant construct; and/or the suture implant construct includes at least first and second implants; and/or the movable inserter is a needle with a piercing end.

A method includes, inter alia, inserting a tissue repair device into a tissue cavity until an end face of a distal end of a fixed outer cannula of the device abuts tissue at or near damaged tissue; sliding a movable inserter of the tissue repair device, that is received in the fixed outer cannula, toward the distal end of the fixed outer cannula; penetrating the tissue with a piercing end of the inserter; and deploying a suture implant construct residing in the movable inserter.

In some embodiments, the method comprises the step of selecting a depth limit that corresponds to a fixed length of the fixed outer cannula; the step of selecting a depth limit includes rotating a depth selector coupled to a proximal end of the fixed outer cannula to a selected depth; the movable inserter remains inside of the fixed outer cannula until the end face of the fixed outer cannula abuts the tissue; the method comprises the step of compressing the tissue with the end face of the distal end of the fixed outer cannula; the step of deploying a suture implant construct includes deploying a first implant of a plurality of implants supported by the movable inserter; and/or the fixed outer cannula remains stationary and flush against the tissue while the inserter penetrates the tissue.

### BRIEF DESCRIPTION OF THE DRAWINGS

FIG. 1 is a perspective view of a tissue repair device, showing an inner moveable inserter outside of the device for clarity.

FIG. 2 is a perspective view of a fixed outer cannula and the moveable inserter of the tissue repair device illustrated in FIG. 1.

FIG. 3 is a partial perspective end view of the inserter illustrated in FIG. 2, showing a suture implant construct associated with the inserter.

FIG. 4 is a partial perspective end view of the inserter illustrated in FIG. 3, the inserter being shown as transparent for clarity in order to illustrate the suture implant construct residing inside of the inserter.

FIG. 5 is a partial perspective end view of the outer cannula, showing the outer cannula as transparent for clarity.

FIG. 6 is a side elevational view of a handle and deployment method for the implants.

FIG. 7 is an enlarged perspective view of one end of the tissue repair device.

FIG. 8 is a cross-sectional view of the one end of the tissue repair device illustrated in FIG. 7.

FIGS. 9A and 9B illustrate a method of tissue repair.

### DETAILED DESCRIPTION

This disclosure generally relates a tissue repair device 10 configured to deliver a suture-implant construct or constructs and methods of repairing damaged tissue. An exemplary suture-implant construct includes one or more stands of suture and one or more implants or anchors. The implants may be any known type of implant or anchor, such as a soft sheath anchor, PEEK anchors, and the like. The tissue repair device 10 may be designed for in-line synchronization and simultaneous movement of a suture implant construct or constructs within an inserter for deploying the same. The tissue repair device 10 is configured to reduce or eliminate damage to tissue or cartilage when inserting the device into a tissue cavity or joint. The tissue repair device 10 is designed to be sturdy and more robust than conventional device to facilitate insertion of the tissue repair device 10 into the tissue cavity during a repair.

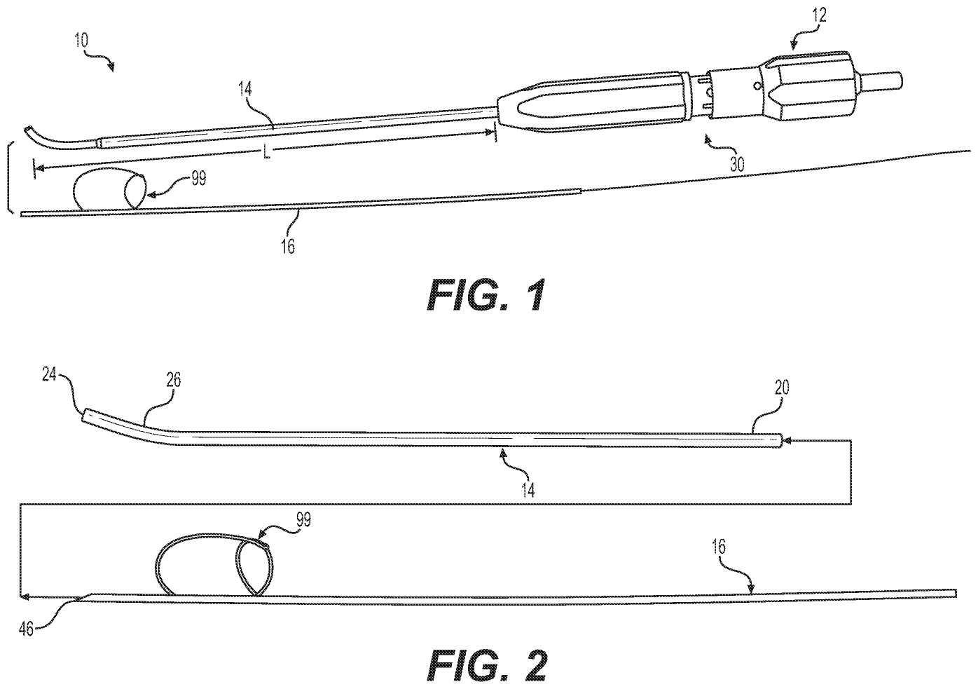

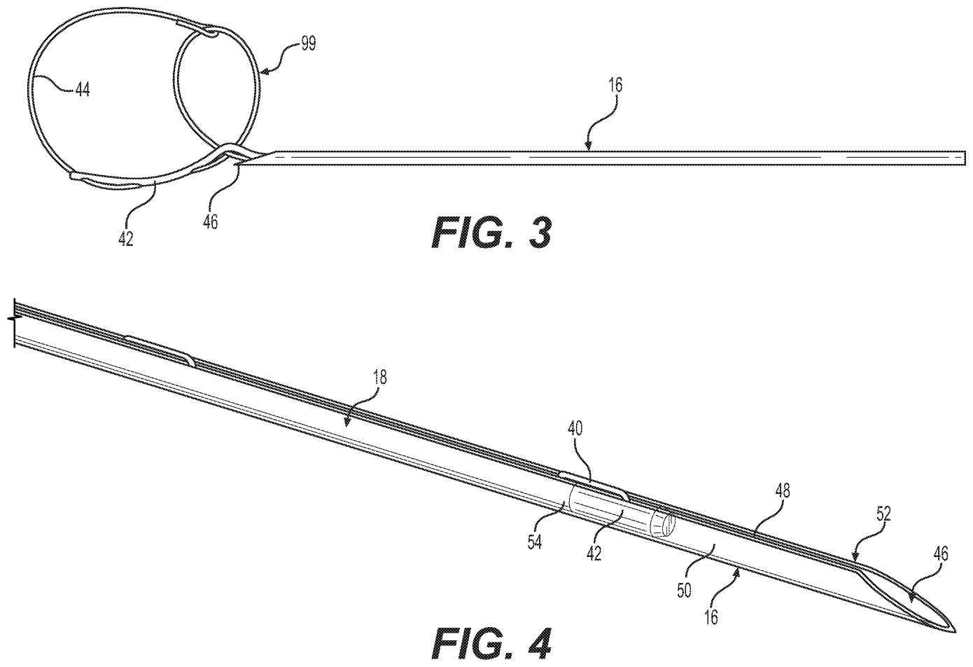

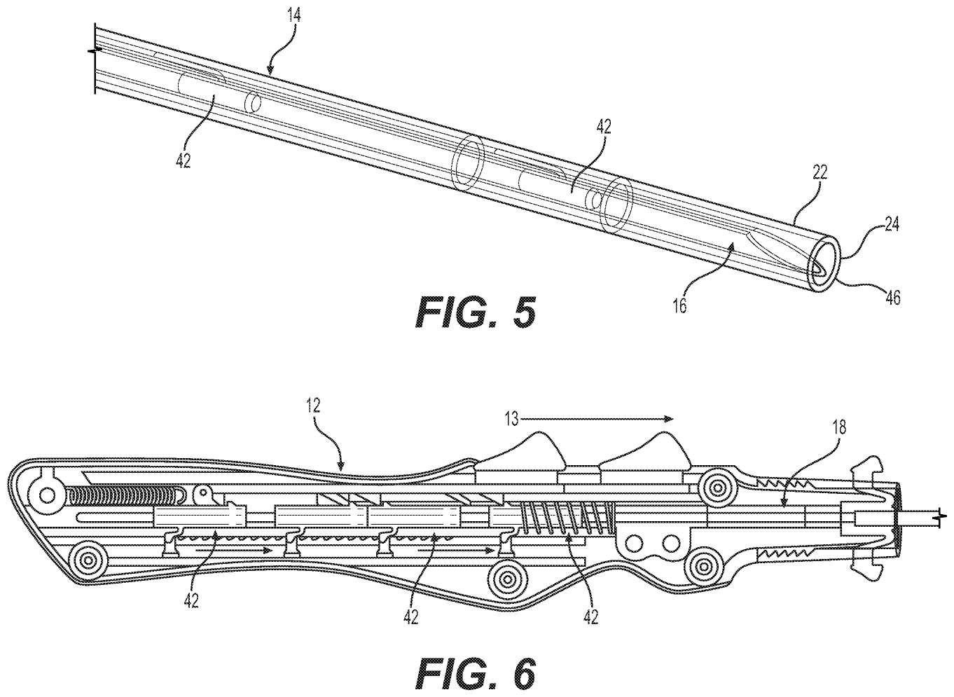

FIG. 1 illustrates an example surgical device 10 for tissue repair. The surgical device 10 generally includes a handle 12, a fixed outer cannula 14 projecting distally from the handle 12 along a longitudinal axis, the fixed outer cannula 14 being fixed to the handle 12, and a moveable inserter 16 received in the outer cannula 14 configured to deploy a suture-implant construct or constructs 99. The moveable inserter 16 is configured to slide with respect to the fixed outer cannula 14 along the longitudinal axis of the cannula 14. An actuator 18 (FIG. 4) may be provided for deploying the suture implant construct 99 through the inserter 16.

The handle 12 may include a trigger 13 (FIG. 6) that is moveable in the distal and proximal directions to move the actuator 18, which itself is moveable within the inserter 16 in the distal and proximal directions. In one example, the handle 12 may include a spring or other biasing element configured to bias the trigger 13 in the proximal direction. In order to move the trigger 13 in the distal direction, a user (i.e., a surgeon) uses his or her thumb, for example, to apply a force to the trigger 13 sufficient to overcome the bias of the spring such that the trigger 13 slides distally. When the user's thumb is released from the trigger 13, the trigger 13 moves proximally back to a resting position under the bias of the spring or other biasing element.

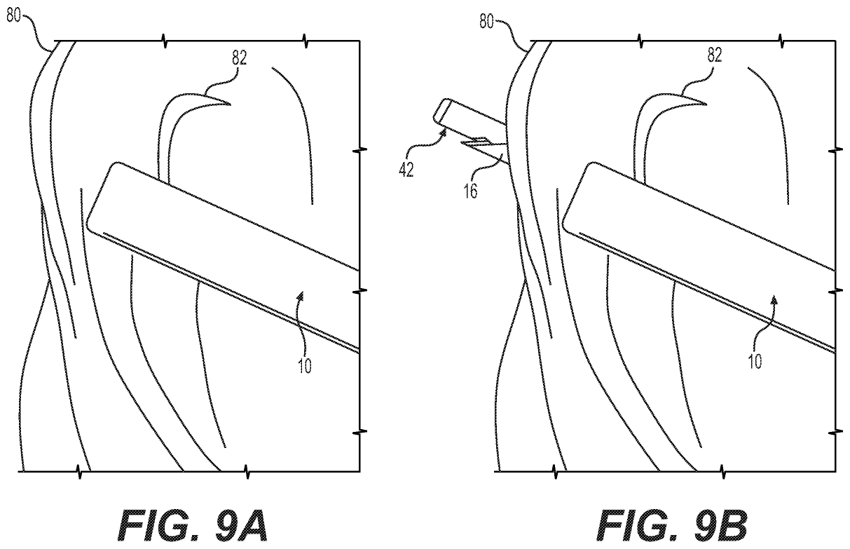

The fixed outer cannula 14 includes a proximal end 20 associated with the handle 12 and an opposite distal end 22 configured to be inserted into a tissue cavity within the body. The fixed outer cannula 14 defines a fixed length L (FIG. 1) which corresponds to an insertion depth limit of the device 10 when inserted into the tissue cavity. An end face 24 of the distal end 22 of the outer cannula 14 defines a depth stop and is configured to abut the tissue 80 (FIGS. 9A and 9B) in the tissue cavity. A tip 26 (FIG. 2) of the cannula's distal end 22 may be substantially flat, relatively sharp, curved, tapered, and/or pointed.

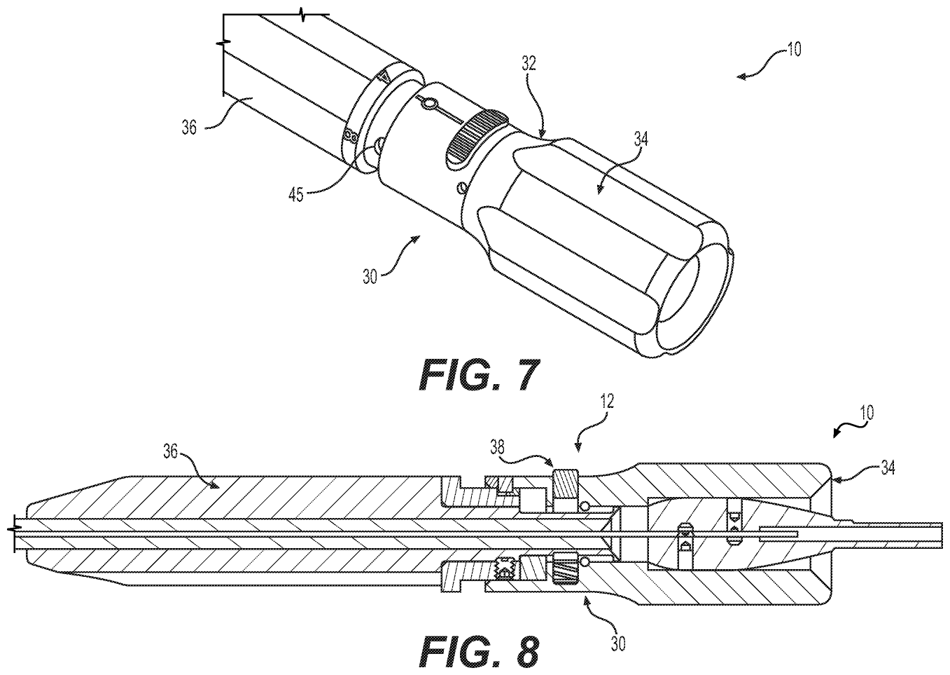

The tissue repair device 10 may be provided with a depth limit selection feature 30 that corresponds to the fixed length L of the outer cannula 14. The depth limit selection feature 30 may be associated with the handle 12 at the proximal end 20 of the outer cannula 14. In an embodiment, the depth limit selection feature 30 comprises a rotating depth selector 32 (FIG. 7) that indexes and acts as a positive stop to control the distance the outer cannula 14 can protrude from the handle 12, i.e. fixed length L. The rotating depth selector 32 may involve rotation of a gripping section 34 of handle 12 over a housing section 36, as seen in FIG. 8. A spring loaded retainer 38 is captured in the gripping section 34 such that when the gripping section 34 is rotated over the housing section 36, the retainer 38 locks into one of the depth selection notches 45, thereby setting the appropriate depth (and fixed length L) determined by the user.

The moveable inserter 16 is received within the fixed outer cannula 14 and is slidable therein. The inserter 16 is configured to be loaded with a suture-implant construct or constructs 99, which may comprise one or more strands of suture 40 and one or more implants 42. The strand or strands of suture 40 may be attached to each implant 42 in any known manner, such as be splicing the strand 40 through splice points in the implant 42, such as with a sheath type implant. An end of the strand may be affixed back to the strand 40 by a knot or other fixation technique to create a loop 44 enclosing a portion of a first implant 42. The loop 44 retains the implant 42 relative to the strand of suture 40 and ensures that the implant does not slide distally off the strand of suture 40.

The suture-implant construct 99 may be referred to as a “soft” construct because it is formed of soft materials such as yarns, fibers, filaments, strings, fibrils, strands, sutures, etc., or any combination of such materials. The soft materials may be synthetic or natural materials, or combinations of synthetic and natural materials, and may be biodegradable or non-biodegradable within the scope of this disclosure. In an embodiment, the suture-implant construct 99 is made exclusively of soft, suture-based materials. The soft materials confer the ability to be inserted into or through tissue (e.g., bone, ligament, tendon, cartilage, etc.) and then bunch together, collapse, expand, and/or change shape to fixate the suture-implant construct 99 relative to the tissue.

In an embodiment, the strand or strands of suture 40 may be any flexible strand suitable for surgical tissue repair. For example, the strand or strands of suture 40 may be one of the following examples: FiberWire®, TigerWire®, or FiberChain® suture, which are each available from Arthrex, Inc. It should be understood, however, that any type of suture may be used, including cored or coreless sutures. In another embodiment, the strand of suture 40 is flat suture, such as FiberTape® or SutureTape® suture, which is also available from Arthrex, Inc. The strand or strands of suture 40 may also be a monofilament suture having barbs. Further, the strand or strands of suture 40 could include any soft, flexible strand of material, and is not limited to suture.

In an embodiment, the inserter 16 is a surgical needle with a piercing end 46. A longitudinal slot 48 may be provided in a wall of the inserter 16 that extends generally parallel to the longitudinal axis of the cannula 14 and provides access to an inside of the inserter 16. The width of the slot 48 may be sized to accommodate the strands or strands of sutures 40 of the construct 99. The slot 48 includes opposing side walls 50 and 52 which serve to guide movement of the actuator 18 in a direction parallel to the longitudinal axis. The slot 48 may extend along a portion of the length of the cannula 14 or alternatively may extend along the entire length of the cannula 14.

The actuator 18 may be a slider that is configured to push and deploy the one or more implants 42 in sequence. In one example, the actuator 18 includes a pushrod or shaft mechanically coupled to the trigger 13 of the handle 12. The actuator 18 is configured to move in the distal and proximal directions in response to corresponding movement of the trigger 13. The actuator 18 may include a relatively smooth superior surface and relatively smooth side surfaces configured to slide relative to the respective side walls 50 and 52 of the slot 48. The actuator 18 can have a deployment mechanism, such as a shuttling rack or the like, which is configured to interact with implants 40 to move them distally within the inserter 16.

A method of using the tissue repair device 10 will now be described with reference to FIGS. 9A and 9B. The surgeon initially determines the appropriate insertion depth limit of the device 10 into the cavity and then fixes the length L (FIG. 1) of the outer cannula 14 based on that determined depth limit. The length L of the outer cannula 14 may be fixed, for example, by using the depth selection feature 30. The construct 99 is loaded into the inserter 16 with a distal end 54 (FIG. 4) of the actuator 18 positioned proximal to the first implant 42, and the loaded inserter 16 is positioned inside the fixed outer cannula 14.

The surgeon inserts the surgical device 10 within the tissue cavity or joint space, in order to deploy the one or more implants 42 in the area adjacent the tear 82 in the tissue 80 in order to close the tear 82 and allow it to heal. The surgeon can insert the device 10 until the end face 24 of the outer cannula 14 abuts the tissue 80 in the cavity, such as on the medial or inside surface of a meniscus. The fixed length L of the cannula 14 ensures that the device 10 is inserted to the proper depth in the tissue cavity and not too deep, which could result in damage to the tissue or cartilage. This avoids the need to move the cannula 14, after that device 10 is already into the tissue cavity, to determine whether the inserter has been inserted to the proper depth, such as is the case in conventional tissue repair devices. In an embodiment, the surgeon can use the end face 24 of the outer cannula 14 to compress the tissue near the tear 82 while conducting the repair.

Once the device 10 is inserted to the proper depth in the tissue cavity, the inserter 16 is moved to slide with respect to the cannula 14 toward distal end 22 in order to penetrate the tissue 80 in a first location where a first implant 42 of the suture implant construct 99 is to be deployed. When in this first position, the fixed outer cannula 14 remains stationary and flush against the tissue as the inserter 16 is penetrating the tissue. The surgeon can penetrate the inserter 16 into, for example, a medial surface of the tissue and out a lateral surface of the tissue. Once the inserter 16 slides and penetrates the tissue 80 at the first location, the surgeon operates the trigger 13 (FIG. 6), which moves the actuator 18 (FIG. 4) distally to selectively deploy the suture-implant construct 99 and its implants 42. The distal end of the actuator 18 does not extend beyond the inserter 16 as the implants 42 are pushed out to the piercing end 46 of the inserter 16.

After the first implant is deployed, the surgeon can remove his or her thumb, for example, from the trigger 13, thereby allowing the trigger 13 and the actuator 18 to move proximally. A second anchor is then moved from a standby position to a deploy position. The surgeon can position the cannula 14 flush against the tissue 80 in a second location adjacent the tear 82, and that is spaced-apart from the first location, and repeat the above process to deploy the second implant. In an embodiment, once the first and second implants are deployed adjacent the tear 82, the strand of suture 40 is tensioned to cinch or tension the implants, thereby closing the tear 82.

It should be understood that terms such as “lateral,” “medial,” “distal,” “proximal,” “superior,” and “inferior” are used above consistent with the way those terms are used in the art. Further, these terms have been used herein for purposes of explanation, and should not be considered otherwise limiting. Terms such as “generally,” “substantially,” and “about” are not intended to be boundaryless terms, and should be interpreted consistent with the way one skilled in the art would interpret those terms.

Although the different examples have the specific components shown in the illustrations, embodiments of this disclosure are not limited to those particular combinations. It is possible to use some of the components or features from one of the examples in combination with features or components from another one of the examples.

One of ordinary skill in this art would understand that the above-described embodiments are exemplary and non-limiting. That is, modifications of this disclosure would come within the scope of the claims. Accordingly, the following claims should be studied to determine their true scope and content.

What we claim is:

1. A tissue repair device, comprising
a fixed outer cannula having a distal end and a proximal end opposite the distal end and a fixed length, the fixed length defining a depth stop; and
a movable inserter received inside of the fixed outer cannula, the movable inserter being slidable with respect to the fixed outer cannula, and the movable inserter being configured to support a suture implant construct that is positioned for deployment.

2. The device of claim 1, further comprising an actuator received in the fixed outer cannula for sliding the movable inserter with respect to the fixed cannula.

3. The device of claim 1, further comprising a depth limit selection feature that corresponds to the fixed length of the fixed outer cannula.

4. The device of claim 3, wherein the depth limit selection feature is associated with a handle at the proximal end of the fixed outer cannula.

5. The device of claim 3 where the depth limit selection feature is a rotating depth selector coupled to the proximal end of the fixed outer cannula.

6. The device of claim 1, wherein the movable inserter includes a longitudinal slot providing access to an inside of the movable inserter.

7. The device of claim 5, wherein the width of the longitudinal slot is sized to accommodate a suture or sutures of the suture implant construct.

8. The device of claim 1, wherein the suture implant construct includes at least first and second implants.

9. The device of claim 1, wherein the movable inserter is a needle with a piercing end.

10. A method of tissue repair, comprising the steps of
inserting a tissue repair device into a tissue cavity until an end face of a distal end of a fixed outer cannula of the device abuts tissue at or near damaged tissue;
sliding a movable inserter of the tissue repair device, that is received in the fixed outer cannula, toward the distal end of the fixed outer cannula;
penetrating the tissue with a piercing end of the inserter; and
deploying a suture implant construct residing in the movable inserter.

11. The method of claim 10, further comprising the step of selecting a depth limit that corresponds to a fixed length of the fixed outer cannula.

12. The method of claim 11, wherein the step of selecting a depth limit includes rotating a depth selector coupled to a proximal end of the fixed outer cannula to a selected depth.

13. The method of claim 10, wherein the movable inserter remains inside of the fixed outer cannula until the end face of the fixed outer cannula abuts the tissue.

14. The method of claim 10, further comprising the step of compressing the tissue with the end face of the distal end of the fixed outer cannula.

15. The method of claim 10, wherein the step of deploying a suture implant construct includes deploying a first implant of a plurality of implants supported by the movable inserter.

16. The method of claim 10, wherein the fixed outer cannula remains stationary and flush against the tissue while the inserter penetrates the tissue.
